(19) United States
(12) Patent Application Publication
(10) Pub. No.: US 2010/0331761 A1
(43) Pub. Date: Dec. 30, 2010

(54) Apparatus and Method for Dermatological Wound Healing

(75) Inventors: Francis A. Papay, Westlake, OH (US); James G. Papay, Elyria, OH (US); Anthony Balsamo, Lumberville, PA (US)

Correspondence Address: TAROLLI, SUNDHEIM, COVELL & TUMMINO L.L.P., 1300 EAST NINTH STREET, SUITE 1700, CLEVELAND, OH, 44114, US

(73) Assignee: The Cleveland Clinic Foundation

(21) Appl. No.: 12/690,786

(22) Filed: Jan. 20, 2010

Related U.S. Application Data
(60) Provisional application No. 61/145,984, filed on Jan. 21, 2009.

(51) Int. Cl. A61N 1/30

(52) U.S. Cl. 604/21; 604/20

(57) ABSTRACT

A method is provided for treating a dermatological wound. A first step of the method includes providing an iontophoretic apparatus. The iontophoretic apparatus includes at least one surface for contacting a surface of the dermatological wound, a current source electrically coupled to the apparatus, a suction mechanism operably coupled to the apparatus, and a source of therapeutic molecules. A second step of the method includes placing the at least one surface of the iontophoretic apparatus into contact with at least a portion of the dermatological wound surface. A third step of the method includes activating the current source so that a plurality of therapeutic molecules is delivered to the dermatological wound. A fourth step of the method includes activating the suction mechanism to remove fluid and/or debris from the dermatological wound. The second, third, and fourth steps of the method are repeated until the dermatological wound is sufficiently healed.

### RELATED APPLICATION

This application claims priority from U.S. Provisional Patent Application No. 61/145,984, filed Jan. 21, 2009, the entirety of which is hereby incorporated by reference.

### TECHNICAL FIELD

The present invention relates generally to an apparatus and method for wound healing, and more particularly to an iontophoretic apparatus and related method for healing dermatological wounds.

### BACKGROUND OF THE INVENTION

Iontophoresis is a needle-free, non-invasive technology for delivering nutrients, medicines, vitamins, minerals, therapeutic agents, drugs, or other bioactive agents through the skin using a small electric current. In general, delivering such medicaments through iontophoresis involves applying an electromotive force that transports ions through the stratum corneum, the outermost layer of skin, and into the dermis, the inner layer of skin comprised of connective tissue, blood and lymph vessels, sweat glands, hair follicles, and an elaborate sensory nerve network.

Compared to popular methods of delivering drugs, such as local skin patches, injections, or oral delivery, there are significant advantages to delivering medicaments through iontophoresis. First, compared to local skin patches, using iontophoresis enhances the skin's permeability, allowing for greater faster drug delivery, higher dose rates, and shorter treatment times. Second, compared to hypodermic injection, iontophoresis is non-invasive and thereby increases patient compliance, avoids painful injections, and reduces the associated risk of infections.

Certain drawbacks exist for using iontophoresis to treat dermatological wounds, however. For example, treating dermatological wounds using iontophoresis can cause localized pH alterations as a result of accumulation of electrolysis products and cellular necrosis. The build-up of such products can then shield bacteria, fungi, etc. in the region from penetration of therapeutic molecules to the proper tissue depth.

### SUMMARY OF THE INVENTION

According to one aspect of the present invention, a method for treating a dermatological wound in a subject is provided. A first step of the method includes providing an iontophoretic apparatus. The iontophoretic apparatus comprises at least one surface for contacting a surface of the dermatological wound, a current source electrically coupled to the apparatus, a suction mechanism operably coupled to the apparatus, and a source of therapeutic molecules for delivery to the dermatological wound. A second step of the method includes delivering the therapeutic molecules to the dermatological wound. A third step of the method includes activating the suction mechanism to remove fluid and/or debris from the dermatological wound. The second and third steps of the method are repeated until the dermatological wound is sufficiently healed.

According to another aspect of the present invention, a method is provided for treating a dermatological wound in a subject. A first step of the method includes providing an iontophoretic apparatus. The iontophoretic apparatus includes at least one surface for contacting a surface of the dermatological wound, a current source electrically coupled to the apparatus, a suction mechanism operably coupled to the apparatus, and a source of therapeutic molecules. A second step of the method includes placing the at least one surface of the iontophoretic apparatus into contact with at least a portion of the dermatological wound surface. A third step of the method includes activating the current source so that a plurality of therapeutic molecules is delivered to the dermatological wound. A fourth step of the method includes activating the suction mechanism to remove fluid and/or debris from the dermatological wound. The second, third, and fourth steps of the method are repeated until the dermatological wound is sufficiently healed.

According to another aspect of the present invention, a method is provided for treating a bed sore in a subject in a subject. One step of the method includes providing an iontophoretic apparatus. The iontophoretic apparatus comprises at least one surface for contacting a surface of the bed sore, a current source electrically coupled to the apparatus, a suction mechanism operably coupled to the apparatus, and a source of therapeutic molecules for delivery to the bed sore. A second step of the method includes placing the at least one surface of the iontophoretic apparatus into contact with at least a portion of the bed sore surface. A third step of the method includes activating the current source so that a plurality of the therapeutic molecules is delivered to the bed sore. A fourth step of the method includes activating the suction mechanism to remove fluid and/or debris from the bed sore. The second, third, and fourth steps of the method are repeated until the bed sore is sufficiently healed.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a process flow diagram illustrating a method for treating a dermatological wound according to one aspect of the present invention.

### DETAILED DESCRIPTION

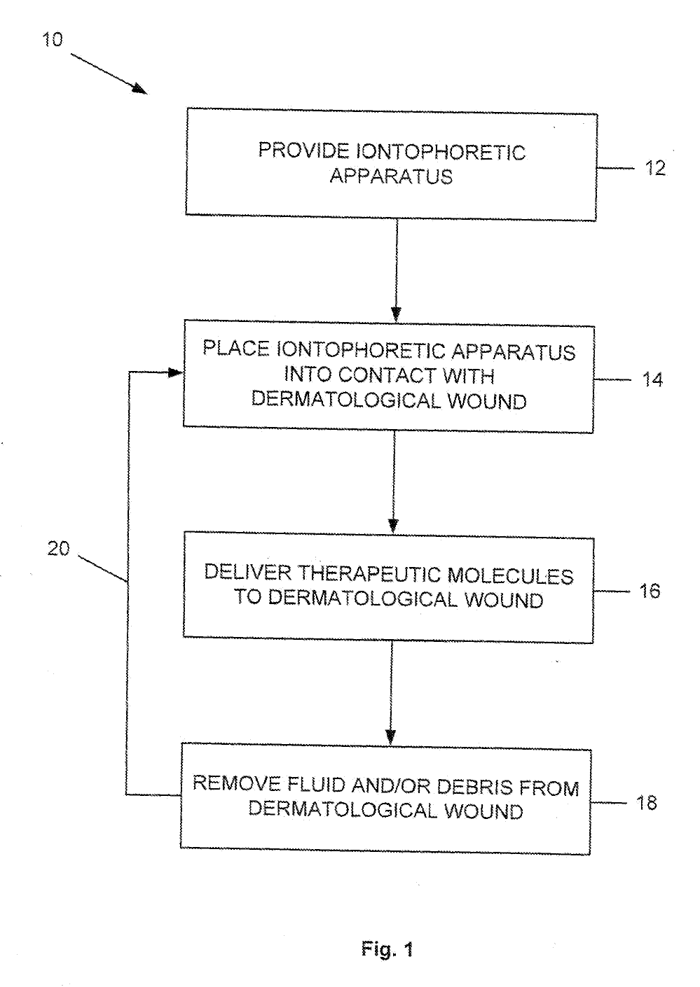

The present invention relates generally to an apparatus and method for wound healing, and more particularly to an iontophoretic apparatus and related method for healing dermatological wounds. The present invention provides an iontophoretic apparatus (not shown) and method 10 (FIG. 1) for treating a dermatological wound in a subject. Although the method 10 will be described below in terms of treating decubital ulcers (“bed sores”), it will be appreciated that any other type of dermatological wound can be treated by the present invention.

Unless otherwise defined, all technical terms used herein have the same meaning as commonly understood by one of ordinary skill in the art to which the present invention pertains.

As used in the context of the present invention, the term “dermatological wound” can refer to damage or loss to any one or combination of skin layers caused by cuts, incisions (including surgical incisions), abrasions, microbial infections, diseases or disorders, necrotic lesions, lacerations, fractures, contusions, burns, and amputations.

As used herein, the term “subject” can refer to any warm-blooded organism including, but not limited to, human beings, pigs, rats, mice, dogs, goats, sheep, horses, monkeys, apes, rabbits, cattle, etc.

As used herein, the terms “treatment” and “treating” can refer to obtaining a desired physiologic, dermatological, or cosmetic effect by the present invention. The effect may be prophylactic in terms of completely or partially preventing a disease, disorder, or symptom thereof and/or may be therapeutic in terms of a partial or complete cure for a disease, disorder, and/or symptom attributable to the disease or disorder. Thus, the terms can cover any treatment of a disorder or disease in a subject, such as: (a) preventing a dermatological wound from occurring in a subject that may be predisposed to the dermatological wound but has not yet been diagnosed as having it; (b) inhibiting a dermatological wound, e.g., arresting its development; and (c) relieving, alleviating, or ameliorating a dermatological wound by, for example, causing regression of the dermatological wound.

As used herein, the term “iontophoresis” can refer to the migration of ionizable therapeutic molecules through a medium driven by an applied low level electrical potential.

FIG. 1 is a process flow diagram illustrating one aspect of the present invention. In FIG. 1, a method 10 is provided for treating a dermatological wound in a subject. The method 10 can be used to treat a variety of dermatological wounds, such as those listed above, as well as fresh injuries and surgical incisions, bed sores, infected and traumatic wounds, and the like. As described in more detail below, the present invention promotes dermatological wound healing by efficiently and effectively removing undesirable byproducts of iontophoresis therapy while also delivering therapeutic molecules to promote healing of dermatological wounds.

As shown in FIG. 1, Step 12 of the method 10 includes providing an iontophoretic apparatus. The iontophoretic apparatus can include any apparatus, device, or member capable of causing the migration of ionizable therapeutic molecules through a medium by an applied low level electrical potential. The design and configuration of the iontophoretic apparatus may vary depending on the place onto which the iontophoretic apparatus is to be delivered and the corresponding treatment regimen. Generally, the iontophoretic apparatus can comprise at least one surface for contacting a surface of the dermatological wound, a current source electrically coupled to the iontophoretic apparatus, a suction mechanism operably coupled to the iontophoretic apparatus, and a source of therapeutic molecules. Other features of the apparatus can include an irrigating catheter for ensuring penetration of therapeutic molecules into a wound. For example, the irrigating catheter could be advanced during operation of the apparatus into a deep wound or a sinus tract to irrigate the wound and ensure deep penetration of therapeutic molecules.

The current source of the iontophoretic apparatus can comprise a first electrically-conductive material, a second electrically-conductive material, and an electrical mechanism capable of providing an electrical current between the first and second electrically-conductive materials. In one example of the present invention, the first electrically-conductive material can comprise a treatment electrode, the second electrically-conductive material can comprise a return electrode, and the electrical mechanism can comprise a low-voltage, DC or AC power source having positive and negative terminals in electrical communication with the treatment electrode and the return electrode, respectively.

One or both of the electrodes can be made from a flexible, electrically-conducting material. For example, materials used to form the treatment electrode can include metals and metal alloys, such as gold, copper, platinum, zinc, or the like. Examples of materials used to form the return electrode can include flexible carbon rubber, carbon-filled fabrics, metal-containing fabrics, or the like. The electrodes can be configured in any suitable way so that electrical current is conducted between the electrodes when the low voltage power source is activated.

The electrodes can be contained in a single housing, such as a transdermal patch or, alternatively, contained in separate housings. The size and shape of the electrodes can be varied as needed. The size and shape of the electrodes usually corresponds to the size and shape of the dermatological wound to be treated. For example, the treatment electrode can have a slightly larger surface area than the area of a dermatological wound. The appropriate size and thickness of the electrodes can be readily determined by those of skill in the art.

One or both of the electrodes can include a source of ionizable therapeutic molecules for delivery a dermatological wound. For example, the treatment electrode can include a pad of absorbent material soaked in a therapeutic composition. The therapeutic composition can comprise any substance or material that includes at least one ionizable therapeutic molecule capable of being delivered to a dermatological wound. Alternatively, the therapeutic composition can be added directly to the treatment electrode once the treatment electrode has been placed at the dermatological wound. Additionally, it will be appreciated that the treatment electrode can also be coated with a therapeutic composition.

In another example of the present invention, the first electrically-conductive material can comprise a first galvanic material, the second electrically-conductive material can comprise a second galvanic material, and the electrical mechanism can comprise a resistive material that separates the first and second galvanic materials. The resistive material can produce a current flow when placed in contact with an electrolytic fluid, such as saline or a bodily fluid (e.g., blood). The first and/or second galvanic materials can also include a therapeutic composition. When the resistive material produces a current flow, ionizable therapeutic molecules can be formed and then driven into a dermatological wound.

The iontophoretic apparatus also includes a suction mechanism for removing fluid and/or debris from a dermatological wound. The suction mechanism can comprise any device, apparatus, and/or member that is operably coupled to the iontophoretic apparatus and that is capable of generating or providing a negative pressure. For example, the suction mechanism can include a vacuum port integrally formed with the iontophoretic apparatus. The vacuum port can extend from the at least one surface of the iontophoretic apparatus to a suction source, such as a pump. Alternatively, the suction mechanism can comprise a seal integrally formed with a vacuum line. The seal can be placed on a dermatological wound to provide a negative pressure over all or a just a portion of the dermatological wound. The suction mechanism can be operated at or below atmospheric pressure (i.e., negative pressure). The suction mechanism can be applied constantly, periodically, or cyclically. As described in more detail below, the suction mechanism can promote dermatological wound healing by removing fluid and/or debris that accumulates at the dermatological wound site.

At step 14 of the method 10, at least one surface of the iontophoretic apparatus is placed into contact with a surface of the dermatological wound. Application of the iontophoretic apparatus to the dermatological wound can be done as soon as possible following an acute injury. Depending on the type of injury, however, application of the iontophoretic apparatus to the dermatological wound may be initiated any time after injury, or whenever deemed medically necessary. Before application of the iontophoretic apparatus to the dermatological wound, cleaning and debridement of the wound may be needed.

After preparing the dermatological wound surface, at least one surface of the iontophoretic apparatus can be placed so that it partly or entirely covers the dermatological wound. Where a subject is suffering from a bed sore, for example, the entire surface of the bed sore can be covered by a surface of the iontophoretic apparatus. When applying the iontophoretic apparatus to the dermatological wound surface, care should be taken to eliminate or reduce the presence of void spaces between the surface of the iontophoretic apparatus and the surface of the dermatological wound (even if filled with a composition), which can result in inadequate treatment at those points.

At step 16 of the method 10, a plurality of therapeutic molecules is delivered to the dermatological wound. The manner in which the therapeutic molecules are delivered to the dermatological wound will depend upon the configuration of the iontophoretic apparatus. Generally, activation of the electrical mechanism will cause the first and second electrically-conductive materials to obtain opposite charge polarities. The opposite charge polarities will then cause the therapeutic molecules in the therapeutic composition to ionize. The ionized therapeutic molecules will then be driven into the dermatological wound as a result of the repulsive force between the electrically-conductive materials and the ionized therapeutic molecules. Where the therapeutic composition contains silver, for example, activation of the electrical mechanism can cause ionized silver ions to migrate into the bed sore(s) of a subject and thereby kill any bacteria present in the bed sore(s).

Depending upon the type of dermatological wound, any one or combination of therapeutic compositions can be used. Examples of such therapeutic compositions can include those containing dermatological agents, antibacterial agents, antifungal agents, anticonvulsant agents, antihypertensive agents, anticancer agents, immunomodulatory agents, antiviral agents, anesthetics, analgesics, tranquilizers, sedatives, muscle relaxants, non-steroidal anti-inflammatory agents, cosmetic agents, biologics, small molecules, polynucleotides, polypeptides and steroids.

More specific examples of such agents can include vitamin A, C, D or E, alpha-hydroxy acids, such as pyruvic, lactic or glycolic acids, beta-hydroxy acids, caffeine, theobromine, lanolin, vaseline, aloe vera, methyl or propyl parban, pigments, dyes and the like for tattooing and make-up effects, estrogen, and other make-up agents, anti-aging agents, pigments, such as iron oxide and titanium oxide) for uses after demiabrading of the skin for tattoo removal, iodine to reduce scar tissue, nutrients, DNA, RNA corticosteroids and -caine type compounds, such as lidocaine in base form, estradiol, progesterone, demegestone, promegestone, testosterone, and their esters, nitro-compounds, such as nitroglycerine, and isosorbide nitrates, nicotine, chlorpheniramine, terfenadine, triprolidine, hydrocortisone, oxicam derivatives, such as piroxicam, ketoprofen, mucopolysaccharides, such as thiomucase, buprenorphine, fentanyl and its analogs, naloxone, codeine, dihydroergotamine, pizotiline, salbutamol, terbutaline, prostaglandins, such as misprostol and emprostil, omeprazole, imipramine, benzamides, such as metaclopramide, scopolamine, peptides, such as growth releasing factor, epidermal growth factor, and somatostatin, cloidine, dihydroxypyridines, such as nifedipine, verapamil, ephedrine, proanolol, metoprolol, spironolactone, thiazides, such as hydrochlorothiazide, flunarizine, syndone imines, such as molsiodmine, sulfated polysaccharides, such as heparin fractions, and salts of such compounds with physiologically acceptable acids and bases.

Either simultaneous with or subsequent to delivery of therapeutic molecules, the suction mechanism is activated at Step 18. Activation of the suction mechanism creates a negative pressure at the dermatological wound to remove any unwanted fluid and/or debris while permitting delivery of additional therapeutic molecules to the dermatological wound. It is known that operation of conventional iontophoresis devices can create unwanted localized pH alterations at the wound site due to accumulation of electrolysis products, cellular necrosis, and build-up of dead tissue. Such alterations can shield microorganisms, such as bacteria and fungi from delivery of the therapeutic molecules and thereby contribute to further wound development. By removing unwanted fluid and/or debris during treatment, an optimal healing environment is created by the present invention.

From the above description of the invention, those skilled in the art will perceive improvements, changes and modifications. For example, delivery of therapeutic molecules and removal of fluid and/or debris from the dermatological wound can be repeated at Step 20 until satisfactory wound healing is observed. Such improvements, changes, and modifications are within the skill of the art and are intended to be covered by the appended claims.

Having described the invention, the following is claimed:

1. A method for treating a dermatological wound in a subject, said method comprising the steps of:
(a) providing an iontophoretic apparatus, the iontophoretic apparatus comprising at least one surface for contacting a surface of the dermatological wound, a current source electrically coupled to the apparatus, a suction mechanism operably coupled to the apparatus, and a source of therapeutic molecules for delivery to the dermatological wound;
(b) delivering the therapeutic molecules to the dermatological wound; and
(c) activating the suction mechanism to remove fluid and/or debris from the dermatological wound;
(d) wherein steps (b)-(c) are repeated until the dermatological wound is sufficiently healed.

2. The method of claim 1, wherein said step of delivering the therapeutic molecules to the dermatological wound further comprises the steps of:
placing the at least one surface of the iontophoretic apparatus into contact with at least a portion of the dermatological wound surface; and
activating the current source so that a plurality of the therapeutic molecules are delivered to the dermatological wound.

3. The method of claim 1, wherein said step of providing an iontophoretic apparatus further includes the step of providing an irrigation catheter.

4. The method of claim 3, wherein said step of activating the suction mechanism further comprises the steps of:
advancing the irrigation catheter into the dermatological wound; and
irrigating the dermatological wound to ensure penetration of therapeutic molecules in the dermatological wound during delivery of the therapeutic molecules.

5. The method of claim 1, wherein said step of activating the suction mechanism is done at least one of constantly, periodically or cyclically.

6. The method of claim 1 further comprising the step of cleaning and debriding the dermatological wound prior to said step of activating the current source.

7. The method of claim 1, wherein said step of activating the suction mechanism is done simultaneous with said step of activating the current source.

8. A method for treating a dermatological wound in a subject, said method comprising the steps of:
(a) providing an iontophoretic apparatus, the iontophoretic apparatus comprising at least one surface for contacting a surface of the dermatological wound, a current source electrically coupled to the apparatus, a suction mechanism operably coupled to the apparatus, and a source of therapeutic molecules for delivery to the dermatological wound;
(b) placing the at least one surface of the iontophoretic apparatus into contact with at least a portion of the dermatological wound surface; and
(c) activating the current source so that a plurality of the therapeutic molecules are delivered to the dermatological wound;
(d) activating the suction mechanism to remove fluid and/or debris from the dermatological wound;
(e) wherein steps (b)-(d) are repeated until the dermatological wound is sufficiently healed.

9. The method of claim 8, wherein said step of providing an iontophoretic apparatus further includes the step of providing an irrigation catheter.

10. The method of claim 9, wherein said step of activating the suction mechanism further comprises the steps of:
advancing the irrigation catheter into the dermatological wound; and
irrigating the dermatological wound to ensure penetration of therapeutic molecules in the dermatological wound during delivery of the therapeutic molecules.

11. The method of claim 8, wherein said step of activating the suction mechanism is done at least one of constantly, periodically or cyclically.

12. The method of claim 8 further comprising the step of cleaning and debriding the dermatological wound prior to said step of activating the current source.

13. The method of claim 8, wherein said step of activating the suction mechanism is done simultaneous with said step of activating the current source.

14. A method for treating a bed sore in a subject, said method comprising the steps of:
(a) providing an iontophoretic apparatus, the iontophoretic apparatus comprising at least one surface for contacting a surface of the bed sore, a current source electrically coupled to the apparatus, a suction mechanism operably coupled to the apparatus, and a source of therapeutic molecules for delivery to the bed sore;
(b) placing the at least one surface of the iontophoretic apparatus into contact with at least a portion of the bed sore surface; and
(c) activating the current source so that a plurality of the therapeutic molecules are delivered to the bed sore;
(d) activating the suction mechanism to remove fluid and/or debris from the bed sore;
(e) wherein steps (b)-(d) are repeated until the bed sore is sufficiently healed.

15. The method of claim 14, wherein said step of placing the at least one surface of the iontophoretic apparatus into contact with at least a portion of the bed sore surface further comprises covering the entire surface of the bed sore with the at least one surface.

16. The method of claim 14, wherein said step of providing an iontophoretic apparatus further includes the step of providing an irrigation catheter.

17. The method of claim 16, wherein said step of activating the suction mechanism further comprises the steps of:
advancing the irrigation catheter into contact with the, bed sore; and
irrigating the bed sore to ensure penetration of therapeutic molecules in the bed sore during delivery of the therapeutic molecules.

18. The method of claim 14, wherein said step of activating the suction mechanism is done at least one of constantly, periodically or cyclically.

19. The method of claim 14 further comprising the step of cleaning and debriding the bed sore prior to said step of activating the current source.

20. The method of claim 14, wherein said step of activating the suction mechanism is done simultaneous with said step of activating the current source.
